(19) United States
(12) Patent Application Publication
(10) Pub. No.: US 2015/0190614 A1
(43) Pub. Date: Jul. 9, 2015

(54) MR-Capable or RF-Capable Medical Guide Wire

(71) Applicant: EPflex Feinwerktechnik GmbH, Dettingen/Erms (DE)

(72) Inventor: Bernhard Uihlein, Dettingen (DE)

(73) Assignee: FPFLEX fEINWERKTECHNIK GmbH

(21) Appl. No.: 14/422,517

(22) PCT Filed: Jan. 9, 2013
(86) PCT No.: PCT/EP2013/050331

§ 371 Date: Feb. 19, 2015

(30) Foreign Application Priority Data
Aug. 20, 2012 (DE) 10 2012 214 785.3

(51) Int. Cl. A61M 25/01; G01R 33/44; A61M 25/09; G01R 33/28

(52) U.S. Cl. CPC A61M 25/0127; G01R 33/286; G01R 33/44; A61M 25/09; A61M 2025/09075; A61M 2025/09108; A61M 2025/09133

(57) ABSTRACT

A medical guide wire is provided having a wire core made, for example, of MR-invisible material and a sheath that surrounds the wire core at least sectionally and so as to be in touching contact therewith. The sheath can have a multilayer structure which has at least two solid material layers and/or fiber layers which are formed by different, MR-invisible plastics materials. The MR marker has at least one MR marker element which is integrated at least partially into the multilayer structure of the sheath or is surrounded thereby.

### BACKGROUND AND SUMMARY OF THE INVENTION

The invention relates to a medical guide wire having a wire core made of MR-invisible material, a sheath that surrounds the wire core at least sectionally and so as to be in touching contact therewith, and an MR marker made of MR-visible material. The invention furthermore relates to a medical guide wire that is suitable for RF (radiofrequency) applications.

An MR marker is understood here to mean a component of the guide wire, enabling the latter to be rendered visible in magnetic resonance imaging applications, MRI or MR applications for short, including nuclear magnetic resonance (NMR) applications. Accordingly, the characteristic “MR-visible” denotes materials which show artifacts that are detectable in such MR applications, i.e. are visible, in contrast to “MR-invisible” materials, which do not show any such artifacts and are therefore not visible in such applications. The guide wire is of course otherwise embodied such that it is suitable for applications of this kind. This usually includes the choice of a nonmetal material for the wire core, typically a plastics material.

MR-visible guide wires have already been proposed a number of times. Thus, the laid-open specification WO 2007/000148 A2 discloses a guide wire which is constructed from one or more rods and a non-ferromagnetic matrix material that surrounds the rods and/or bonds them together. Each particular rod consists of one or more nonmetal filaments and a non-ferromagnetic matrix material that surrounds the latter and/or bonds them together, said matrix material being doped with an MR marker. Suitable nanoparticles are proposed as the MR marker and an epoxy resin is proposed as the matrix material.

Similarly, the laid-open specification WO 2009/141165 A2 proposes a guide wire which has at least one rod made of a poorly electrically conductive material which is constructed from a matrix material and nonmetal filaments such as glass fibers, ceramic fibers, natural fibers or plastics fibers. The surface region of the instrument body formed in this way is provided with an immobilized active MR marker made of particular chemical substances, or the matrix material of the rod-like instrument body is doped with a passive MR marker, for example in the form of special metal particles which can act at the same time as X-ray markers.

The laid-open specification WO 01/95794 A1 discloses a guide wire which is formed by a tube made of polymer material, into the hollow interior of which an MR marker has been introduced, wherein the latter can act at the same time as an X-ray marker or an X-ray marker can additionally be provided. Salts and oxides of dysprosium are proposed in particular as the MR marker material. In a variant embodiment, a thin glass-fiber thread extends through the hollow interior of the polymer tube, leaving a radial clearance, and serves as a holder for a number of axially spaced-apart MR marker elements.

In order to make instruments for invasive medicine visible, the laid-open specification DE 10 2008 006 402 A1 proposes a coating with a ferrofluid which contains paramagnetic iron oxide nanoparticles.

The patent DE 10 2006 020 402 B3 discloses a guide wire for a catheter designed for brain examinations, wherein the guide wire comprises magnetic nanoparticles suspended in liquid and/or in powder form, in order to be able to move said guide wire to a target by applying external magnetic fields.

Many conventional medical guide wires consist of a wire core and a single-layer sheath, wherein the wire core is formed from a material having greater flexural rigidity than the sheath, such that it determines the flexural rigidity of the guide wire as a whole. For this reason, the wire core is frequently tapered toward the front, distal end, in order to reduce the flexural rigidity of the guide wire in this area of use. Often, the sheath is also additionally provided with a, for example hydrophilic surface coating adapted to the particular application.

The patent DE 10 2005 022 688 B4 discloses a guide wire in which the wire core is surrounded by a sheath that is more flexurally rigid than the wire core only in a shaft section adjoining a distal section. In this type of guide wire, the flexural rigidity of the shaft section is consequently determined by the sheath, which consists for example of a PEEK (polyether ether ketone) material or a polyimide material, and not by the wire core. The more flexible wire core therefore does not necessarily need to be tapered in the distal region in order to provide the desired lower flexural rigidity for the distal section compared with the shaft section. Furthermore, in this guide wire, MR markers in the form of filling balls or cavities, which can be doped with suitable foreign substances, have been introduced into the distal sleeve of the wire core.

The invention is based on the technical problem of providing a medical guide wire of the type mentioned at the beginning, which is further improved with regard to flexural rigidity behavior and/or MR visibility and/or usability in RF applications compared with the abovementioned prior art, and can be manufactured with relatively little effort and high functional reliability.

The invention achieves this object according to a first aspect by the provision of a guide wire comprising a wire core made of an MR-invisible material, a sheath that surrounds the wire core at least sectionally and so as to be in touching contact therewith, and an MR marker made of MR-visible material. The sheath has a multilayer structure which contains two or more solid material layers and/or fiber layers, lying one on top of another, of different, MR-invisible plastics materials. Not included in this case is an optional, typically very thin, for example hydrophilic surface coating of the conventional type, with which the sheath can be provided on its outer side. The MR marker has at least one MR marker element which is integrated at least partially into the sheath or is surrounded thereby.

As a result of this specific multilayer structure, the sheath of the wire core can be matched optimally to the requirements of the particular application. In particular, it is clear that, if desired, with a given guide wire thickness on account of the multilayer structure, the sheath can be realized as desired with much higher flexural rigidity compared with the wire core, while retaining the other properties required for guide wires, and so the flexural rigidity of the guide wire is determined virtually exclusively by the sheath and not by the wire core in the section in which the sheath is present. For example the choice of a very hard, brittle material for one of the solid material layers and of a very tough, strong material for another of the solid material layers can contribute thereto. Consequently, the wire core does not need to be designed to achieve correspondingly high flexural rigidity in the sheathed section, but can be optimized with regard to other characteristics. The MR marker element integrated at least partially in the multilayer sheath or surrounded thereby ensures a desired MR visibility of the guide wire in the corresponding region.

In a development of the invention, the sheath layer structure has at least two solid material layers and at least one fiber layer which is formed by a fabric material or fiber material. This can further contribute to achieving desired high flexural rigidity in particular in the case of comparatively thin guide wires.

In a development of the invention, the fact that the sheath has at least two fiber layers having different axial fiber pitches and/or fiber winding directions contributes to high flexural rigidity.

In a development of the invention, the wire core is formed as a wire strand made of a plurality of individual wires that are connected together in a cord- or strand-forming manner. The individual wires are for example monofilament plastics threads or individual wire strands made of plastics material. In a further configuration, the MR marker has an MR-visible material which has been introduced into intermediate spaces between the individual wires of the wire core strand formed in this way. This realizes an MR-visible region of the guide wire in the vicinity of the core, without the wire core itself needing to be manufactured in an MR-visible manner to this end.

In an advantageous development of the invention, the sheath surrounds the wire core at least in a shaft section adjoining a distal section, but not in the distal section, and is embodied with greater flexural rigidity than the wire core. Therefore, in the shaft section, the sheath determines the flexural rigidity of the guide wire. In the distal section, the more flexible wire core can determine the flexural rigidity, such that the distal section remains more flexible overall than the shaft section, as is desired for many guide wire applications. It is favorable here that, for this purpose, the wire core does not necessarily need to be tapered in the distal section, thereby saving corresponding manufacturing effort.

In a configuration of the invention, the solid material layers of the sheath are manufactured from flexurally rigid plastics materials, such as ABS (acrylonitrile butadiene styrene), PEEK, PET (polyethylene terephthalate), Ultramid and/or epoxy resin materials. This realization is suitable in particular for guide wires which are intended to have relatively high flexural rigidity in the region of the sheath.

In a configuration of the invention, the fiber layers are formed from glass fibers, aramid or Kevlar fibers and/or polyester fibers. To this end, a single fiber or a set of a number of parallel fibers can be applied to or wound on the particular substrate, or the fibers can be braided to form a fabric which is designed to form the layer in question.

In a development of the invention, the MR marker contains an MR marker element which is embedded in one of the sheath layers or between two of the sheath layers or between the wire core and the adjacent sheath layer. From a production point of view, this presents favorable options for providing MR visibility of the guide wire in the region of the sheath.

In a further configuration, a plurality of alternatives that are not mutually exclusive lend themselves to the realization of such an MR marker element. Thus, MR-visible particles can be embedded in one or more of the solid material layers. Alternatively, one or more MR line elements can be embedded in the sheath, for example a line element that is continuous along the axial length of the sheath in a rectilinear or helical manner or with some other profile, or a set of a number of line elements that are axially shorter than the sheath and are arranged with or without an offset in the circumferential direction and with or without an axial offset and with identical or different lengths. Furthermore, such an MR marker element can be formed by one of the fibers of a fiber layer in question, to which end the fiber accordingly contains an MR-visible material, for example by doping of MR-visible particles into the fiber material or by production of the fiber from an MR-visible material or by coating the fiber with an MR-visible material. If required, the MR marker element can also contain one or more MR line elements which are embodied as length measuring marker elements and as a result support a length measuring application in the guide wire.

According to a further aspect of the invention a medical guide wire is provided comprising a wire core, a sheath that surrounds the wire core with touching contact in a shaft section, and a sleeve that surrounds the wire core with touching contact in a distal section. An MR-visible, wire-like or tubular auxiliary element made of a non-magnetic material is arranged in a distal guide wire section. With this auxiliary element, the MR-visibility of this distal guide wire section can be enhanced in a targeted manner. In addition, depending on the embodiment of the auxiliary element, the elastic properties of this distal guide wire end section can be improved or influenced in a targeted manner, for example in order to achieve particular shaping properties, such as achieving an angled distal end region or a J-shaped distal tip of the guide wire. To this end, the auxiliary element can consist for example of a non-magnetic metal material.

In an advantageous configuration, the auxiliary element can comprise an auxiliary element wire which surrounds the wire core in a helical manner and/or extends with an axial main component alongside and along the wire core, and/or an auxiliary element tube which surrounds the wire core in a corresponding axial section.

In a further configuration of this aspect of the invention, the MR-visibility of the auxiliary element is provided or increased in that it is doped and/or coated with an MR marker material.

According to a further aspect of the invention a medical guide wire is provided comprising a wire core. The wire core is surrounded, at least in a shaft section adjoining a distal section, by an electrically insulating sheath which contains a multilayer structure with two or more solid material layers and/or fiber layers, lying one on top of another, of different plastics materials. This electrically insulating design makes the guide wire very readily suitable for RF applications, wherein, if required, the wire core can also consist of a metal material, such as a superelastic nickel titanium alloy.

In an advantageous realization of this aspect of the invention, this guide wire that is suitable for

RF applications additionally has the features of the MR-capable guide wire according the other two aspects of the invention mentioned above, and is in this way suitable both for RF and for MR applications.

### BRIEF DESCRIPTION OF THE DRAWINGS

Advantageous embodiments of the invention are illustrated in the drawings and described in the following text. In the drawings:

FIG. 1 shows a shortened longitudinal sectional view of a guide wire having a three-layer shaft-side sheath,

FIG. 2 shows a cross-sectional view along a line II-II in FIG. 1,

FIG. 3 shows a partially sectional perspective view of the guide wire from FIG. 1,

FIG. 4 shows a shortened longitudinal sectional view of a guide wire having a five-layer sheath,

FIG. 5 shows a cross-sectional view along a line V-V in FIG. 4,

FIGS. 6 to 10 show partially sectional perspective views of guide wire variants having an eight-layer sheath and differently introduced MR marker elements,

FIG. 11 shows a perspective view of an MR marker element framework in a further guide wire variant,

FIG. 12 shows a shortened longitudinal sectional view of a guide wire variant having a distal auxiliary element wire,

FIG. 13 shows a view corresponding to FIG. 12 for an auxiliary wire variant having mushroom ends,

FIG. 14 shows a view corresponding to FIG. 12 for an auxiliary wire variant having a distal ring end,

FIG. 15 shows a view corresponding to FIG. 12 for an auxiliary wire variant having a proximal ring end,

FIG. 16 shows a view corresponding to FIG. 12 with an additional ring element for the auxiliary element wire,

FIG. 17 shows a view corresponding to FIG. 12 for a variant having a helical auxiliary element wire,

FIG. 18 shows a view corresponding to FIG. 17 for a variant having an additional axial auxiliary element wire section,

FIG. 19 shows a side view of an auxiliary element wire that is usable in the variants in FIGS. 12 to 18,

FIG. 20 shows a view corresponding to FIG. 19 for a further auxiliary wire variant,

FIG. 21 shows a view corresponding to FIG. 12 for a guide wire variant having a distal auxiliary element sleeve over the wire core,

FIG. 22 shows a view corresponding to FIG. 21 with a further auxiliary element sleeve variant,

FIG. 23 shows a view corresponding to FIG. 21 with yet another auxiliary element sleeve variant,

FIGS. 24 to 29 each show a side view of different usable auxiliary element sleeves for distally surrounding the wire core,

FIG. 30 shows a longitudinal sectional view of an auxiliary element sleeve having MR-visibly doped sleeve material,

FIG. 31 shows a cross-sectional view along a line XXXI-XXXI in FIG. 30,

FIG. 32 shows a cross-sectional view corresponding to FIG. 31 for an auxiliary element sleeve variant with MR-visible surface doping,

FIG. 33 shows a side view of an auxiliary element sleeve variant with partial MR surface doping,

FIG. 34 shows a side view corresponding to FIG. 33 for an auxiliary element sleeve variant with two different MR surface dopings,

FIG. 35 shows a side view of an auxiliary element sleeve variant made of a braided material, and

FIGS. 36 to 38 each show a side view of further auxiliary element sleeve variants with different incisions.

### DETAILED DESCRIPTION OF THE DRAWINGS

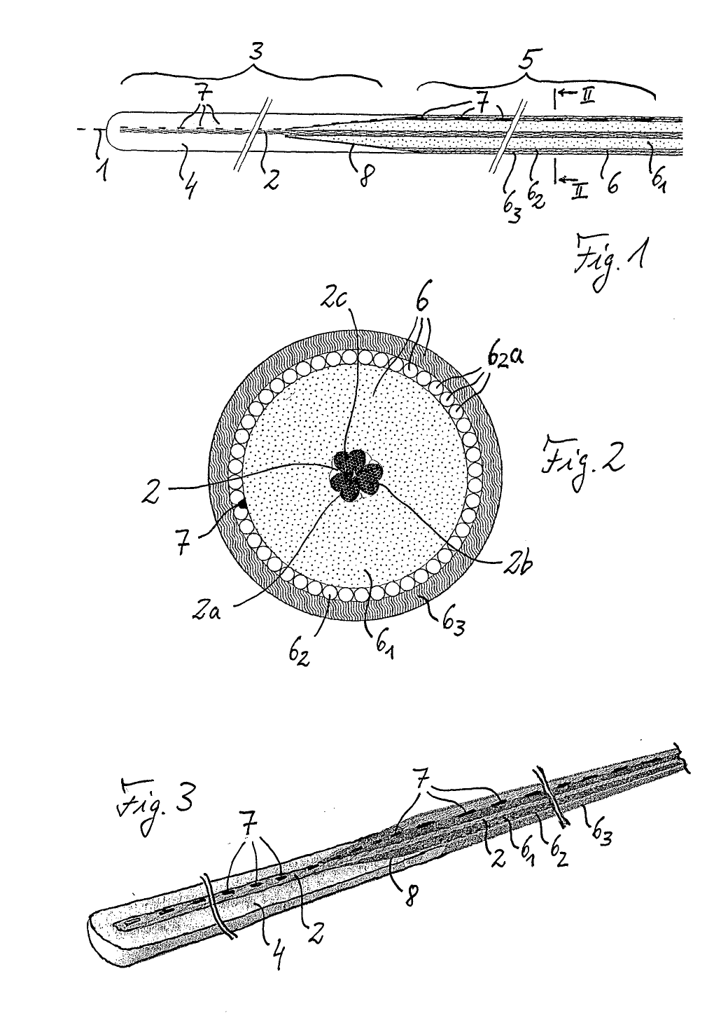

The guide wire as shown in FIGS. 1 to 3 has, as central element along its longitudinal center axis 1, a wire core 2 which is surrounded by a flexible sleeve 4 in a front, distal guide wire section 3 and by a more flexurally rigid sheath 6 in an adjoining shaft section 5. In particular, the sheath 6 has greater flexural rigidity than the wire core 2, and so the flexural rigidity of the guide wire in the shaft region 5 is determined by that of the sheath 6. By contrast, the distal sleeve 4 consists of a material that is much more flexible than the shaft sheath 6, and so the flexibility of the distal guide wire section 3 is greater than in the region of the shaft sheath 6. Since the flexural rigidity of the guide wire in the shaft region 5 is determined by the sheath 6, the wire core 2 can be designed as a whole for achieving a desired flexural rigidity of the distal section 3 without to this end necessarily having to be designed differently in the distal section 3 than in the shaft section 5. Thus, in the example shown, the wire core 2 has the same diameter throughout the distal section 3 as in the shaft section 5. Therefore, the need to grind down the wire core 2 in the distal section 3 or to produce it in some other way with a smaller diameter than in the shaft section 5 in order to achieve lower flexural rigidity for the distal section 3 than for the shaft section 5 is optionally dispensed with.

In the example shown, the wire core 2 is formed by a stranded material made of three individual wires 2a, 2b, 2c, which are for their part in each case in turn manufactured as wire cords or wire strands, and extends in one piece from the distal end to a proximal, rear guide wire end 9. Alternatively, the individual wires can also be realized as monofilament wire sections. Similarly, in alternative embodiments, the wire core 2 can consist as a whole of a monofilament wire section or a complex wire mesh. In each case, the wire core 2 consists of an MR-invisible material, preferably of a relatively elastic and tough, high-strength plastics material, wherein any such material that is known per se to a person skilled in the art for this application purpose is usable.

The sheath 6 has a multilayer structure, specifically, in the example shown, a three-layer structure having an inner solid material layer 6₁, an outer solid material layer 6₃ and an intermediate fiber layer 6₂. The two solid material layers 6₁, 6₃ preferably consist of different materials or material components. Suitable materials therefor are in particular flexurally rigid materials such as ABS, PET, Ultramid and/or epoxy resin plastics materials which can optionally be provided with fillers, wherein in principle all materials which are known per se to a person skilled in the art for this application purpose can come into consideration in turn for use therefor, too. Thus, for example a relatively tough, strong material can be used for one of the two solid material layers 6₁, 6₃, and a relatively hard, brittle material can be used for the other. In any case, the materials for the solid material layers 6₁, 6₃ of the sheath 6, as well as the material for the wire core 2, are MR-invisible materials.

It should be mentioned at this point that in the guide wire according to the invention, the successive layers of the sheath fit closely together with touching contact in the radial direction and the innermost layer fits closely to the wire core with touching contact, as can be seen clearly in FIG. 2 for the example there having the wire core 2 and the three sheath layers 6₁, 6₂, 6₃. Optionally, the outermost sheath layer, in FIG. 2 the layer 6₃, can be provided with a surface coating for example of a hydrophilic type, as is known per se to a person skilled in the art. Such a surface coating is not in the present case considered to be a layer of the sheath 6 but to be an optional, additional external coating.

In order to make the guide wire suitable for use in MR applications, it is provided with an MR marker. In the example shown in FIGS. 1 to 3, this MR marker consists of a plurality of strip-like MR marker elements 7 which are provided along the entire guide wire length. In the distal region 3, they are located in intermediate spaces of the wire strand core 2 or are embedded in the sleeve 4, adjacent to the wire core 2. In the shaft section 5, they are integrated into the sheath 6, specifically into the fiber layer 6₂ in the example shown. In the example in FIGS. 1 to 3, the MR marker elements 7 are arranged in an axially spaced-apart manner along a line, but alternatively, any other desired arrangements are also possible, for example those with a mutual offset in the guide wire circumferential direction. For the MR marker strips 7, use can be made in turn of any conventional, MR-visible material which is known per se to a person skilled in the art for this application purpose.

Advantageously, the MR marker elements 7 can be designed in a distinguishably different manner in the shaft section 5 surrounding the sheath 6, on the one hand, and in the distal region 3 free of the sheath 6, on the other hand, for example with a shorter length and smaller axial spacing in the distal region 3 and longer length and larger spacing in the shaft section 5. In the example shown, the arrangement line of the MR marker elements 7 extends in a longitudinal plane of the guide wire. Alternatively, this arrangement line can also extend in a helically wound manner. In corresponding embodiments, the MR marker elements 7 are embodied as length measuring markers which allow a length measurement to be carried out on the guide wire via the detection of the MR marker elements 7. To this end, the spacings between the successive MR marker elements 7 and/or their axial extents each have a predetermined, defined length. This defined length of the MR marker elements 7 and their spacings along the arrangement line can in this case be selected, if desired, to be different in the distal region 3 than in the shaft section 5. In corresponding embodiments of the invention, in addition to the MR marker elements 7 arranged along a line, provision can be made of further, substantially axially extending MR marker strips of this type which, in order to form corresponding marker rings, are arranged in a manner offset with respect to one another in the circumferential direction and axially for example at the level in each case of one of the MR marker elements located along the arrangement line, wherein the marker rings for their part are preferably positioned at regular axial spacings from one another. Thus, for example every n-th MR marker element 7 which is located on the arrangement line can be supplemented by additional marker strips of the same type, which are arranged in a manner offset thereto in the circumferential direction, to form a marker ring of this type, where n is any desired selectable integer greater than one. This supports the use of such an MR marker for the mentioned length measurement.

For the fiber layer 6₂, for example glass fibers, aramid or Kevlar fibers or polyester fibers are suitable. In the example shown, the fiber layer 6₂ consists of a single-layer, gapless arrangement of fibers 6₂a arranged in a parallel manner alongside one another, said fibers 6₂a being arranged in a manner extending in the axial direction. Alternatively, the fibers 6₂a can be wound around the solid material layer arranged therebeneath in an obliquely extending or helical manner in a selectable winding direction and with a selectable axial fiber pitch.

In the example shown, the distal sleeve 4 does not adjoin the shaft sheath 6 abruptly in the axial direction, but rather with the formation of a continuous, conical transition 8. From a production point of view, this specific embodiment of the guide wire can be realized for example with relatively little effort in that the wire core 2 is initially provided along its entire length with the multilayer sheath 6, subsequently has material removed in the distal section 3, forming a conical taper in the transition region 8, and the distally exposed wire core 2 is provided with the sleeve 4, wherein the latter adjoins the sheath 6 preferably in an externally aligned manner in the transition region 8. This design results in a more uniform transition, depending on the axial extent of the transition region 8, from the greater rigidity, brought about by the sheath 6, of the guide wire region 5 to the lower rigidity of the distal section 3. The initially complete surrounding of the wire core 2 with the sheath 6 can take place for example by corresponding conventional extrusion and fiber-winding operations. It goes without saying that, in alternative embodiments that are not shown, other types of the transition between the distal sleeve 4 and the shaft-side sheath 6 can be provided, for example an abrupt or multistage transition.

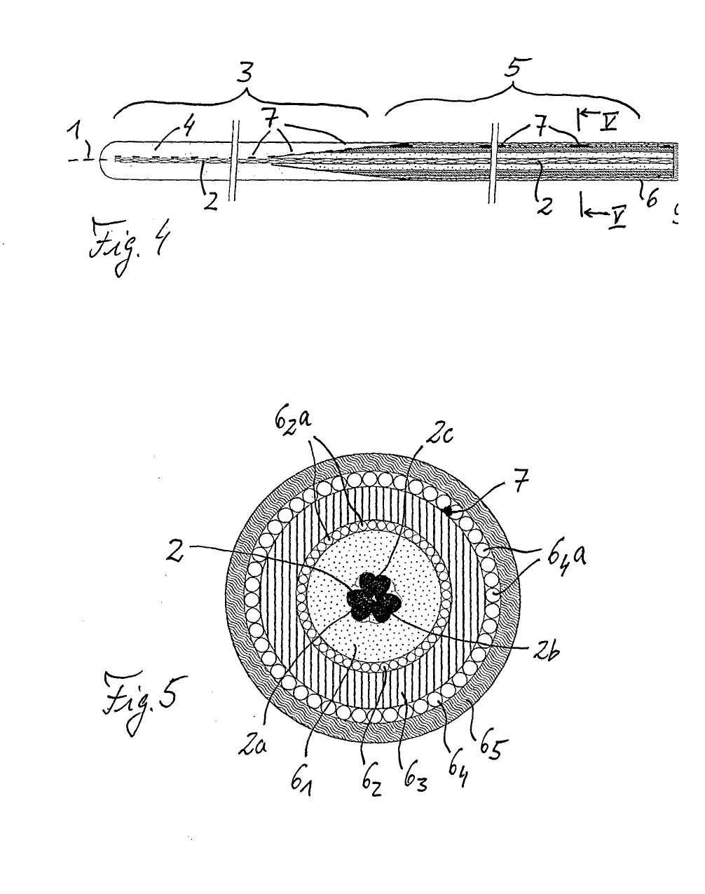

FIGS. 4 and 5 illustrate a variant of the guide wire from FIGS. 1 to 3, said variant differing therefrom merely in the number of layers for the shaft-side sheath 6, wherein, for the sake of easier understanding, identical reference signs have been used for identical and functionally equivalent elements, and in this respect reference can be made to the above description for the exemplary embodiment in FIGS. 1 to 3.

As can be gathered from FIGS. 4 and 5, in this guide wire, the sheath 6 has a five-layer structure having an innermost solid material layer 6₂, an adjoining first fiber layer 6₂, an adjoining middle solid material layer 6₂, an adjoining second fiber layer 6₄ and an outer solid material layer 6₅, which for its part can optionally be provided with a for example hydrophilic external surface coating (not shown). For the solid material layers 6₂, 6₃, 6₅ and the fiber layers 6₂, 6₄, the same materials are in turn usable as are specified above for the relevant layers in the exemplary embodiment of FIGS. 1 to 3. For the three solid material layers 6₂, 6₃, 6₅, three different materials can be used, but alternatively two or all three of the three solid material layers are made of the same material. The two fiber layers 6₂, 6₄ can consist of the same or of different fiber materials. In the example shown, both fiber layers 6₂, 6₄ are in turn formed from a monolayer of individual fibers 6₂a, 6₄a located alongside one another in a touching manner in the circumferential direction, wherein identical or different fiber materials can be used for the two layers 6₂, 6₄. Preferably, the two fiber layers 6₂, 6₄ differ in terms of their fiber winding directions and/or their axial fiber pitches. With a given material and a given thickness for the different sheath layers 6₁ to 6₅, this makes it possible to achieve flexural rigidity of the sheath 6 that is as high as desired.

In the exemplary embodiment in FIGS. 4 and 5, in the same way as in the exemplary embodiment in FIGS. 1 to 3, a number of MR-visible MR marker strip elements are arranged in a line along the entire guide wire length. In the distal section 3, they are embedded in the single-layer sleeve 4 with touching contact with the wire core 2, and in the shaft section 5 they are integrated into the second fiber layer 6₄, i.e. the associated arrangement line extends, as can be seen in FIG. 4, axially along the wire core 2 in the distal region, then with a radial component as far as the second fiber layer 6₄ in the conical transition region 8, and then axially again as far as the rear guide wire end 9.

FIGS. 6 to 10 illustrate different variants of guide wires having MR markers integrated in different ways into the sheath, wherein an in each case eight-layer sheathing is considered only by way of example and wherein identical reference signs are again used for identical or functionally equivalent elements, as for the examples in FIGS. 1 to 5, to the above description of which reference can be made in this respect.

The guide wires in FIGS. 6 to 9 each have a wire strand core 2 made of three individual wire strands 2a, 2b, 2c that are twisted together, said wire strand core 2 being surrounded in the shaft section by a more flexurally rigid sheath of which the eight-layer structure comprises five solid material layers and three fiber layers. Specifically, from inside to outside, these are a first solid material layer 6₁ surrounding the wire core 2 with touching contact, a first fiber layer 6₂, a second solid material layer 6₃, a second fiber layer 6₄, a third solid material layer 6₅, a third fiber layer 6₆, a fourth solid material layer 6₇ and an outer, fifth solid material layer 6₈. As indicated in the drawing, the five solid material layers consist preferably of five different materials, but alternatively only of two to four different materials which differ for example in terms of their hardness or brittleness and/or in terms of their toughness or strength, or all five solid material layers consist of the same material. Thus, layers having different hardnesses and strengths can be combined with one another as desired in order to provide a desired rigidity behavior and other desired properties for the guide wire.

It goes without saying that in alternative embodiments any desired other number of solid material layers can be selected. Similarly, rather than with the three fiber layers shown, the solid material layers can be combined with any desired other number of fiber layers. In the embodiment shown, each fiber layer is located between two adjacent solid material layers, but in alternative embodiments any desired other sequence of solid material layers and fiber layers is usable, for example including two successive fiber layers.

With regard to the materials for the solid material layers and fiber layers, reference can be made to the statements given for the examples in FIGS. 1 to 5. In this case, the materials of the solid material layers can, if required, be provided with additional fillers, as is known per se to a person skilled in the art. The fiber layers can, if required, additionally differ in terms of their winding directions and/or fiber pitches. Thus, in the realization shown in FIGS. 6 to 10, the first fiber layer 6₂ has an axial fiber course, while the fibers of the second fiber layer 6₄ are wound in a helical manner, as is symbolized additionally by way of a residual winding 6₄′ that is also shown to this end in these sectional structural illustrations. The third fiber layer 6₆ is likewise wound in a helical manner but with a considerably larger pitch.

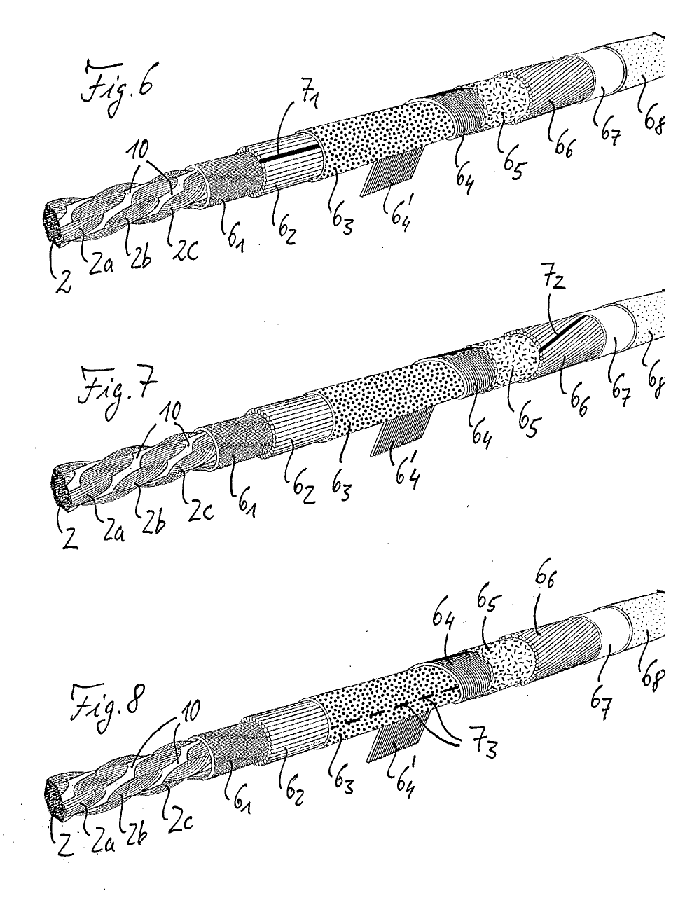

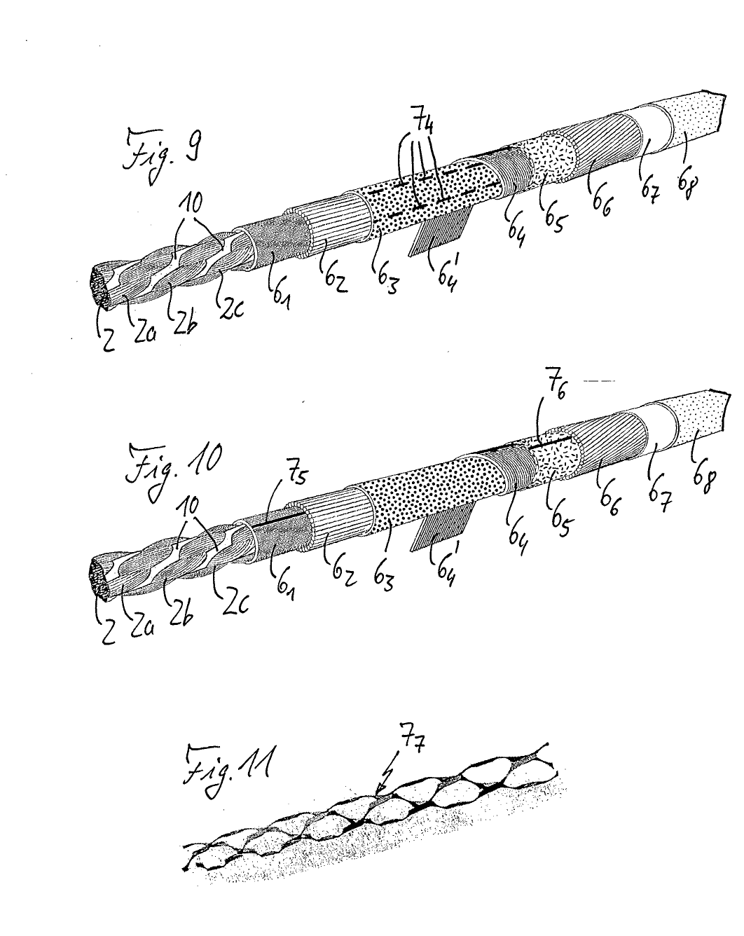

FIGS. 6 to 10 illustrate, as representatives of numerous further options for making the guide wire in the region of the sheath MR-visible, a number of variants in which linear MR marker elements are integrated into the sheath along the axial length of the latter. In the example in FIG. 6, to this end one of the fibers of the first fiber layer 6₂ is manufactured from an MR-visible material and as a result represents an MR marker strip 7₁ that extends axially in this layer 6₂. In the example in FIG. 7, in an analogous manner, one of the fibers of the third fiber layer 6₆ is manufactured from an MR-visible material and as a result represents an MR marker strip that extends helically in this layer 6. In the example in FIG. 8, a number of shorter MR marker strips 7₃ are embedded in the second solid material layer 6₃ in a spaced-apart manner along an axially extending line, or are arranged on the outer side of said second solid material layer 6₃. In the example in FIG. 9, two circumferentially spaced-apart rows of such shorter MR marker strips 7₄ that are axially spaced apart from one another are each embedded in the second solid material layer 6₃ along an axially extending line, or are arranged on the outer side of said second solid material layer 6₃. In the example in FIG. 10, two MR marker strips 7₅ and 7₆ that are continuous along the length of the sheath are embedded into the first solid material layer 6₃ and the third solid material layer 6₅, respectively, in an axially extending manner, or are arranged on the outer side of said solid material layers 6₃ and 6₅, respectively. It goes without saying that, depending on the requirements and the application case, further MR marker elements can be introduced into the various layers of the sheath and the alternatives shown in FIGS. 6 to 10 can be combined as desired.

In addition or as an alternative to such integration of one or more MR marker elements into the sheath, the MR marker can, according to the invention, also comprise MR-visible material which is introduced into intermediate spaces of a wire core realized as a strand. FIG. 11 shows a corresponding MR marker framework 7₇ as is present when an intermediate space 10, as remains between the individual wires within the first solid material layer 6₁ of the sheath in the guide wire variants in FIGS. 6 to 10, is filled in this way with MR-visible material. For better perceptibility, all the other guide wire components have been omitted.

FIGS. 12 to 38 illustrate further embodiments of the invention, specifically those in which a wire-like or tubular auxiliary element made of an MR-visible, non-magnetic material, for example a corresponding metal material, is arranged in a targeted manner in a distal guide wire section, in order to enhance the MR-visibility thereof and/or to deliberately influence the elastic properties thereof. Unless specified to the contrary in the following, the guide wires mentioned with regard to FIGS. 12 to 38 correspond in terms of their constituent parts, functions and properties to the guide wires mentioned above with regard to FIGS. 1 to 11, and so reference can be made in this respect to the above explanations thereof. Furthermore, in each case identical reference signs are used again, here too, for identical and functionally equivalent elements for easier understanding.

The guide wire shown in a shortened manner in FIG. 12 has, like the guide wire in FIGS. 1 to 3, a wire core 2 which is surrounded by a flexible sleeve 4 in a front, distal guide wire section 3 and by a flexurally rigid sheath 6 in an adjoining shaft section 5, as explained in more detail above with regard to FIGS. 1 to 3. In this case, FIG. 12 also shows an example in which the distal sleeve 4 that consists of the more flexible material adjoins the shaft sheath 6 in the axial direction, forming a continuous, conical transition 8.

In addition, the guide wire in FIG. 12 has a wire-like auxiliary element 11 which is embedded in the distal sleeve 4 and extends substantially axially alongside the wire core 2 from the distal end thereof into the conical transition region 8 of the sleeve 4, wherein, in the conical transition region 8, it follows the frustoconical cone with a corresponding radial component in addition to the axial component. The auxiliary element wire 11 consists of an MR-visible, non-magnetic material, preferably a corresponding metal material, such as a superelastic nickel titanium alloy, or an MR-visible plastics material, for example a plastics material which has been doped with an MR marker material. By way of this auxiliary element wire 11, the MR-visibility specifically of the distal end section 3 of the guide wire can be enhanced. Furthermore, the elastic properties of the distal guide wire section 3 can be influenced in a targeted manner, for example so as to provide a desired shape for the distal tip of the guide wire, such as a J-shaped tip form or some other angling of the distal guide wire end.

In the guide wire in FIG. 12, depending on the requirements, the sheath 6 can have a conventional single-layer structure or a multilayer structure, as is described above with regard to FIGS. 1 to 11.

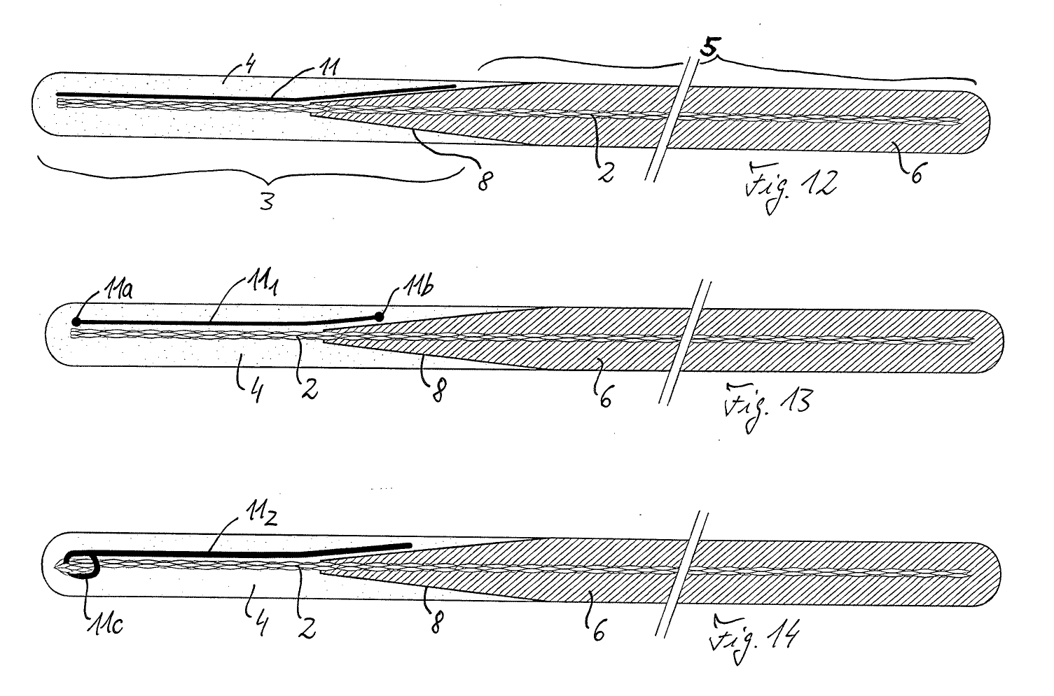

The guide wire shown in FIG. 13 differs from that shown in FIG. 12 by way of a modification of the auxiliary element wire. Specifically, use is made here of an auxiliary element wire 11₁ which is provided with a mushroom-shaped thickening 11a, 11b at both ends. These terminal thickenings 11a, 11b support secure fixing of the auxiliary element wire 11₁ in the embedding, surrounding distal sleeve 4.

The guide wire shown in FIG. 14 contains, as a variant of the one shown in FIG. 12, a modified auxiliary element wire 11₂, at the distal end of which there is formed a wire bend or wire loop 11c with which it is fastened to the distal end of the wire core 2, as shown. To this end, the wire bend 11c engages in and through the fiber mesh or strand material from which the wire core 2 is formed. In this way, the wire-like auxiliary element 11₂ is held with its distal end securely at the distal end of the wire core 2.

FIG. 15 shows a guide wire variant having an auxiliary element wire 11₃, which, in contrast to the guide wire in FIG. 12, is bent at its proximal end into a wire bend or wire loop 11d with which it is placed around the truncated cone of the transition cone 8, this in turn promoting secure fixing of the auxiliary element wire 11₃ in the embedding core sleeve 4.

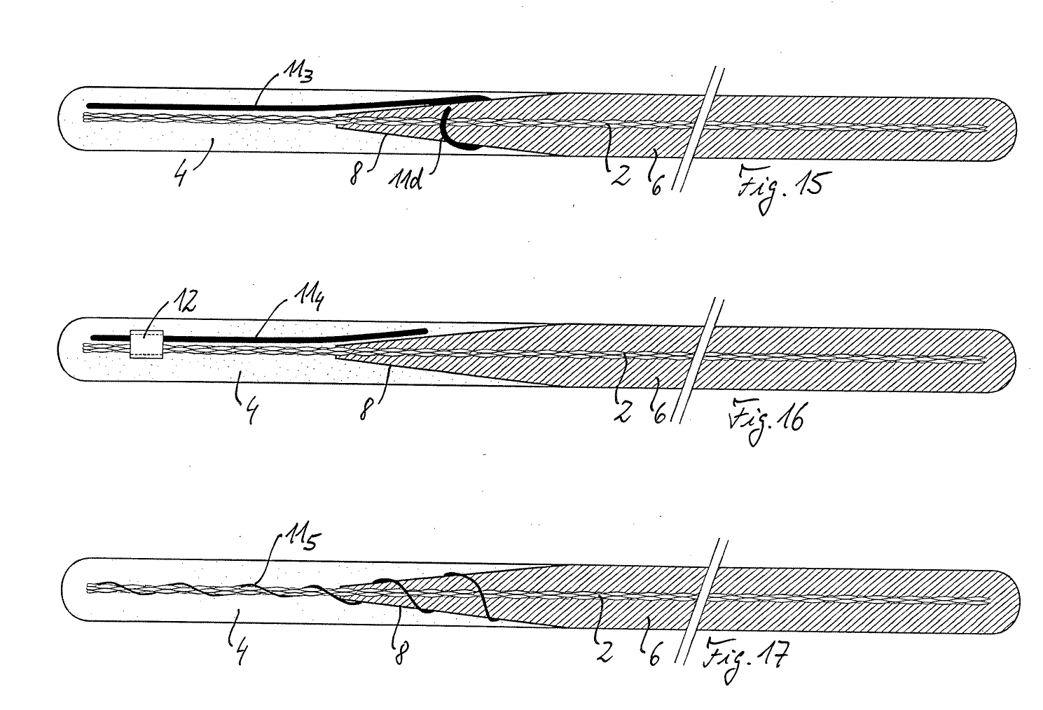

In a guide wire variant shown in FIG. 16, provision is made of an auxiliary element wire 11₄ of the type in FIG. 12, wherein here the auxiliary element wire 11₄ is fixed to the wire core 2 by means of an additional retaining ring 12 in order to retain it securely in the embedding core sleeve 4. Alternatively, it is also possible for a plurality of such retaining rings to be provided axially alongside one another. Each particular retaining ring 12 can consist for example of gold, platinum, tungsten or some other metal and as a result act as an MR marker and as an X-ray visible marker.

FIG. 17 shows, as a further variant, a guide wire having an auxiliary element wire 11₅ which extends helically around the wire core 2 and the transition cone 8. This realization, too, contributes to securely retaining this auxiliary element wire 11₅ in the embedding core sleeve 4.

FIG. 18 shows a guide wire variant which corresponds to a combination of the auxiliary elements in FIGS. 12 and 17. A linear wire section 11₆ of the type of the wire section 11 in FIG. 12 serves there, in combination with a wire section 11₇ extending helically around the wire core 2 and the linear wire section 11₆, as auxiliary element for enhancing the MR-visibility of the distal guide wire section. This accordingly combines the advantages of the variants shown in FIGS. 12 and 17.

In order to realize the wire sections provided as MR-visible auxiliary elements 11 to 11₇ in the guide wires in FIGS. 12 to 18, various alternatives are appropriate depending on the requirements and application case. Thus, for example solid wire sections or wire strands having a cross section that is constant along the length thereof, for example a circular cross section, are usable. Alternatively, the wire cross section can be modified over the wire length in order to achieve desired bending properties of the guide wire. Thus, in one exemplary embodiment, the auxiliary element wire has a diameter that decreases conically in the distal direction, with the result that gradually decreasing flexural rigidity can be set for the distal guide wire end section.

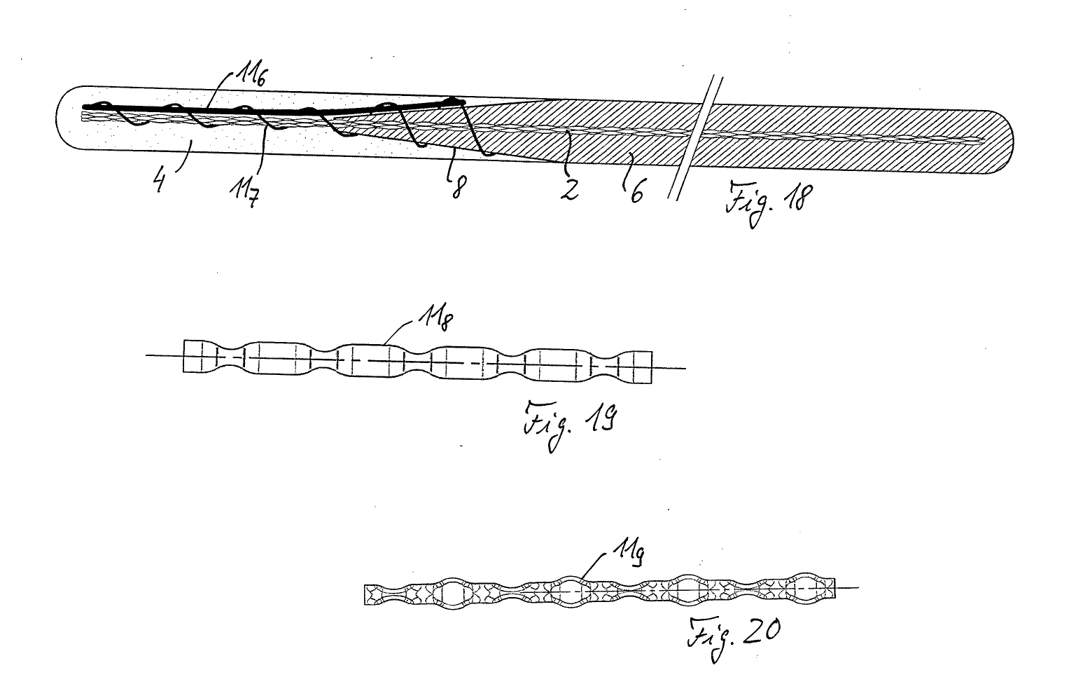

FIG. 19 shows a realization of an auxiliary element wire section 11₈ having a wire diameter that varies in an alternating manner between a minimum diameter value and a maximum diameter value in the axial direction. This auxiliary element wire 11₈ can be manufactured for example by sectional circumferential grinding of a solid unwrought wire section having a constant diameter. FIG. 20 shows an alternatively usable auxiliary element wire 11₉ having a diameter that is periodically variable in the axial direction and can be produced for example by a corresponding wire pressing operation.

Instead of or in addition to a wire-like auxiliary element, as explained above, provision can be made of an MR-visible sleeve-like or tubular auxiliary element made of a non-magnetic material in a distal guide wire section in order to enhance the MR-visibility of said section and if required influence the elastic properties, i.e. bending properties, thereof in a desired manner. To this end, the tubular auxiliary element surrounds the wire core in the distal section in question. Like the wire-like auxiliary elements, the tubular auxiliary elements can also be formed from a suitable MR-visible plastics material or non-magnetic metal material. Depending on the requirements, the wire-like or tubular auxiliary elements can be manufactured from corresponding wire material, strand material, thread material, braided material or tube material. FIGS. 21 to 38 illustrate various guide wire variants having such auxiliary element sleeves or auxiliary element tubes in the distal guide wire section.

FIG. 21 shows, again in a shortened illustration, a guide wire having such an MR-visible auxiliary element tube 12 instead of the auxiliary element wire 11 in FIG. 12, wherein the guide wire in FIG. 21 otherwise corresponds to that in FIG. 12. As shown, the auxiliary element tube 12 surrounds the wire core 2 in the distal end region thereof as far as the transition cone 8 of sleeve 4 and sheath 6. In this example, the auxiliary element tube 12 consists of a uniform tube section. The auxiliary element tube 12 is, as shown, embedded in the flexible distal core sleeve 4, wherein the embedding material also fills the annular gap between the wire core 2 and surrounding auxiliary element tube 12.

FIG. 22 illustrates a variant of the guide wire from FIG. 21 having a modified auxiliary element tube 12₁ which is manufactured from a tube section in which circumferentially opposite incisions 13 that are offset axially with respect to one another have been introduced in the radial direction, said incisions 13 each extending approximately as far as the tube center, i.e. with an approximately half circumferential length. In order to influence the bending behavior of the guide wire in a targeted manner in its distal end section, the incisions 13 can be selected in a suitable manner, for example, as shown, at an axial spacing apart that decreases in the distal direction, such that flexural rigidity that decreases in the distal direction arises overall for the auxiliary element tube 13 and consequently for the distal guide wire section.

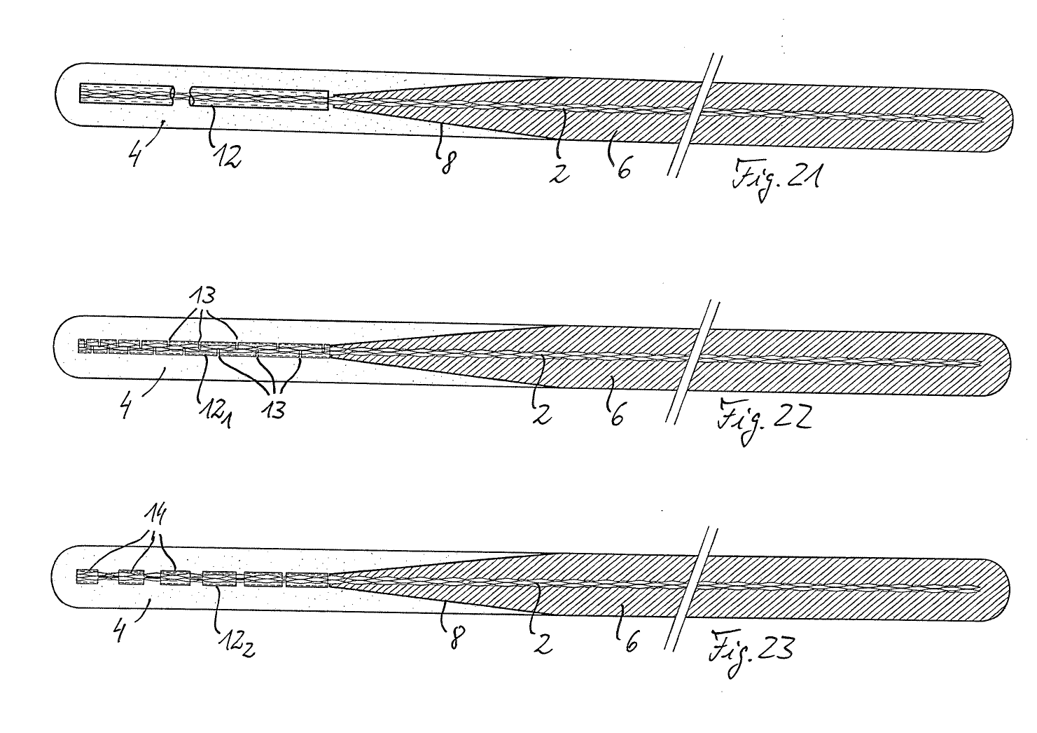

FIG. 23 shows a guide wire variant in which provision is made of an auxiliary element tube 12₂ made of individual tube sections 14 which are embedded in the distal sleeve 4 in a loosely connected-together manner or in a manner that is not shown, and surround the wire core 2. In the example shown, in order to achieve flexural rigidity that decreases in the distal direction, the tube sections 14 are arranged with a length that decreases and a spacing that increases in the distal direction.

FIGS. 24 to 29 show various possible realizations of auxiliary element tubes that are usable according to the invention and are modified with regard to influencing flexural rigidity. Thus, FIG. 24 shows an auxiliary element tube 12₃ which, like the auxiliary element tube 12₁ in the guide wire of FIG. 22, is provided with incisions 13 that are axially offset and are located circumferentially opposite one another in an alternating manner. In this example, the incisions 13 are introduced at an equal axial spacing apart, so that they produce a uniform increase in the flexibility, i.e. a uniform decrease in the flexural rigidity, for this auxiliary element tube 12₃. As an alternative, FIG. 25 shows an auxiliary element tube 12₄ similar to the one in FIG. 24, wherein, however, the incisions 13 have been introduced here with a decreasing axial spacing apart in the distal direction. This leads to reduced flexural rigidity of this auxiliary element tube 12₄ and thus of the distal guide wire section in which the auxiliary element tube 12₄ is employed. This corresponds to the use of the analogous auxiliary element tube 12₁ in the guide wire of FIG. 22.

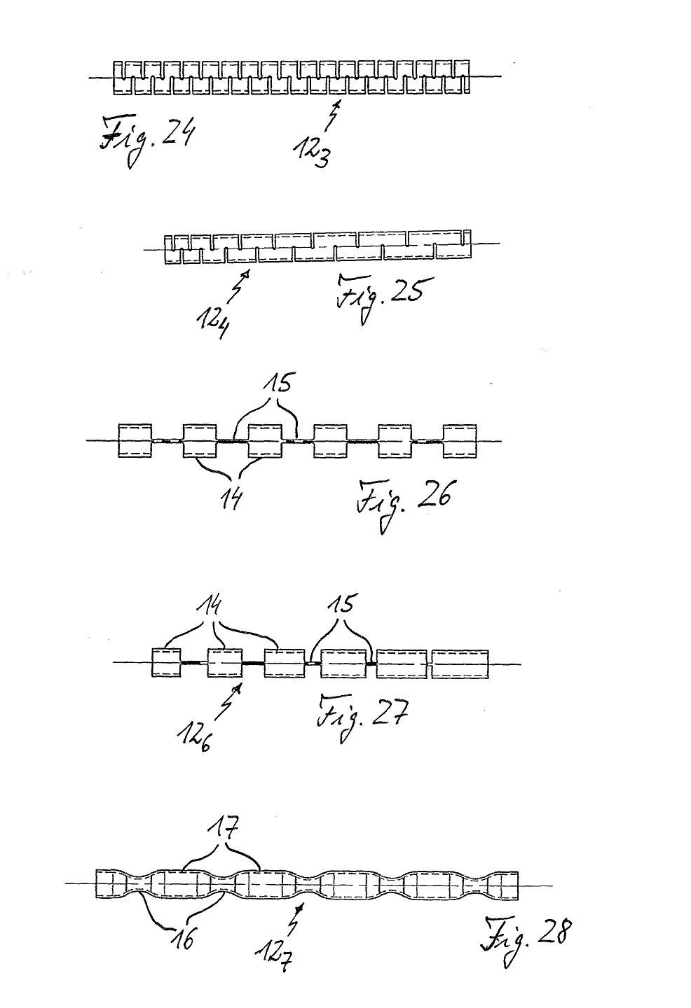

FIG. 26 shows an auxiliary element tube 12₅ which, similarly to the auxiliary element tube 12₂ in the guide wire of FIG. 23, is formed from individual ring segments 14 which are arranged in a successive manner in the axial direction. In this example, the ring segments 14 are connected together via narrow axial crosspieces 15, and so the auxiliary element tube 12₅ forms a one-piece component which can be cut out of a homogeneous tube section for example by way of a suitable cutting process. This results again in decreased flexural rigidity for the auxiliary element tube 12₅ compared with the uncut one-piece tube section.

FIG. 27 shows an auxiliary element tube 12₆ that is modified compared with FIG. 26, and wherein the tube segments or tube sections 14 are arranged with a successively decreasing length and increasing axial spacing apart in the distal direction. This auxiliary element tube 12₆ thus corresponds to the auxiliary element tube 12₂ employed in the guide wire of FIG. 23.

FIG. 28 shows a modified auxiliary element tube 12₇ that has regions 16 with a small diameter and regions 17 with a larger diameter in a sectionally alternating manner. Of course, the regions having the small diameter 16 are dimensioned such that the tube diameter is still greater than the diameter of the wire core there, and so the relevant distal end section of the wire core can be received in the auxiliary element tube 12₇.

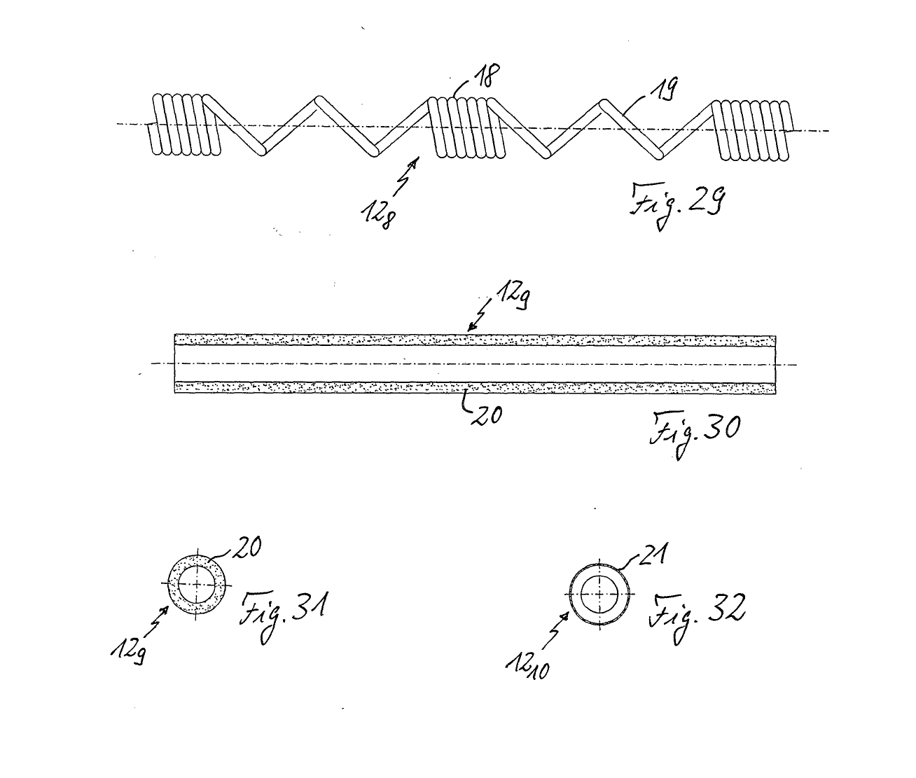

FIG. 29 shows an auxiliary element tube 12₈ which is formed by a helical spring section. In this case, in order to influence the flexural rigidity behavior in a targeted manner, regions 18 in which adjacent coils rest against one another alternate with more flexible regions 19 in which the coils are spread out and as a result are axially spaced apart. It goes without saying that, as an alternative, any other desired helical spring configurations are possible for this type of auxiliary element tube, for example a uniform helical spring section or a helical spring section having a coil spacing that increases successively or in a stepwise manner in the distal direction.

In those guide wire variants in which the wire-like or tubular auxiliary element is not uniform in the axial direction but has a variation, for example the variants having a helical auxiliary element wire or the variants having an auxiliary element tube made of axially successive tube segments or tube sections or of helical spring sections having different coil spacings, this property of visibility that varies accordingly in the axial direction can be used in MR applications or else in X-ray applications for spacing measurements, i.e., as a result of this property, the precise position of the distal guide wire section provided with the wire-like or tubular auxiliary element can be detected very easily and be exploited for spacing measurements.

FIGS. 30 and 31 show a tubular auxiliary element 12₉ which is formed from a uniform tube section that consists of a basic material 20 doped with an MR marker material so as to be MR-visible. This ensures good MR-visibility of a distal guide wire section when this auxiliary element tube 12₉ is employed there for example in the manner of the auxiliary element tube 12 in FIG. 21.

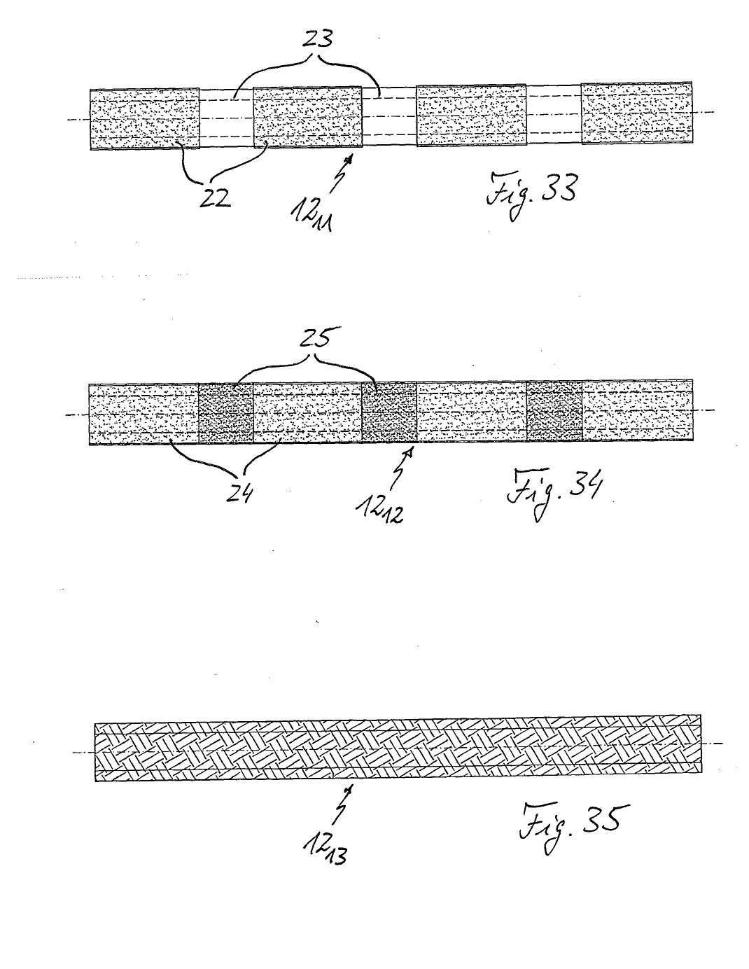

FIG. 32 illustrates a variant of the auxiliary element tube 12₉ from FIGS. 30 and 31 in the form of an auxiliary element tube 12₁₀ which has surface doping or a surface coating 21 made of an MR marker material, instead of the bulk doping in the example of FIGS. 30 and 31.

FIG. 33 illustrates a variant of the auxiliary element tube 12₁₀ in the form of an auxiliary element tube 12₁₁ which, rather than being provided with the surface MR marker doping or MR marker coating over the entire surface of its outer side, is provided only partially therewith. Specifically, the partial coating in this example contains individual tube sections 22 which have been doped or coated so as to be MR-visible and which are arranged at an axial spacing apart, wherein undoped or uncoated regions 23 remain between them.

FIG. 34 illustrates a modification of the auxiliary element tube 12₁₁ from FIG. 33 in the form of an auxiliary element tube 12₁₂ in which, in the axial direction, regions 24 of a first surface MR marker coating or MR marker doping alternate in the axial direction with regions 25 of a second MR marker doping or MR marker coating different from the first. With the variants in FIGS. 33 and 34, as required, the abovementioned spacing measurements and guide wire position determinations are again possible, exploiting the MR-visibility, varying in the axial direction, of the auxiliary element tubes 12₁₁, 12₁₂.

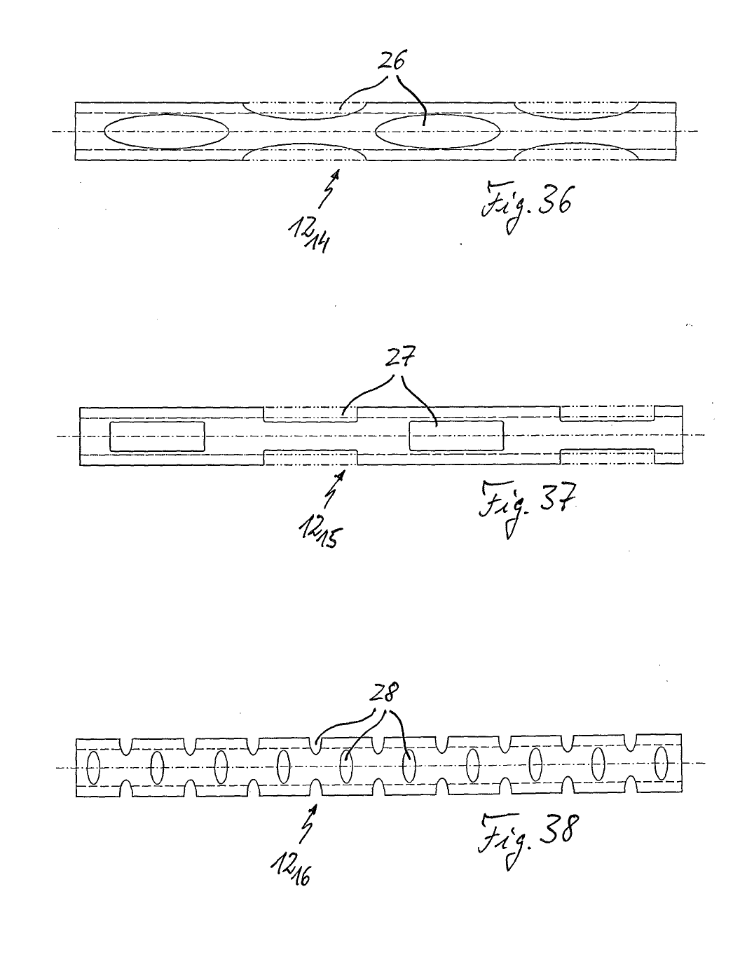

As mentioned, each particular auxiliary element tube can consist of a superelastic nickel titanium alloy, some other non-magnetic metal or from a plastics material that has been doped so as to be MR-visible or alternatively has not been doped. In this case, rather than a uniform, homogeneous structure of the auxiliary element tube made of the relevant material, production from a mesh of such non-magnetic metal or plastics materials is also possible. FIG. 35 shows a corresponding auxiliary element tube 12₁₃ which is formed from such a woven flexible tube material.

FIGS. 36 to 38 illustrate further advantageous configurations of the auxiliary element tubes that are usable according to the invention and are provided with different incisions with which the flexural rigidity can be influenced in the desired manner and in addition a better connection of the auxiliary element tube to the distal section, accommodated therein, of the wire core can be achieved.

Specifically, FIG. 36 shows an auxiliary element tube 12₁₄ which is provided with oval incisions 26 that have been introduced into the tube casing in each case in mutually opposite pairs in the circumferential direction and so as to be offset through 90° in a manner following one another in the axial direction.

FIG. 37 shows a similar auxiliary element tube 12₁₅ in which rectangular incisions 27 have been introduced into the tubular casing instead of the oval incisions 26 in the example of FIG. 36.

FIG. 38 shows an auxiliary element tube 12₁₆ in which oval incisions 28 have been introduced into the tubular casing, said oval incisions extending transversely to the axial direction of the tube 12₁₆ instead of in the tube axial direction like the oval incisions 26 in the example of FIG. 36.

As the numerous examples with reference to FIGS. 12 to 38 make clear, the invention provides a large variety of MR-visible, wire-like or tubular auxiliary elements made of non-magnetic material with which a distal guide wire section can be equipped in order to enhance its MR-visibility and/or to influence its bending properties in a desired manner. In addition, it should be mentioned that, if required, the shaft sheath 4 and/or the distal sleeve 2 can be provided with a marking pattern which can be used in order to detect longitudinal and/or rotational movements of the guide wire and/or to measure lengths. To this end, use can be made in particular of marking patterns as are described in the patents DE 102 43 261 B4 and DE 102 55 030 B4 and in the laid-open specification WO 2009/112048 A1.

The embodiments of the invention that have been described thus far with regard to the figures represent guide wires which can be used not only for MR applications but also for RF applications. For the latter applications, it is not absolutely necessary for the guide wire to have an MR marker and for the wire core thereof to consist of an MR-invisible material. Rather, in this case, the wire core can also consist for example of a superelastic nickel titanium alloy which is surrounded by the multilayer shaft sheath 6 made of electrically insulating material, which ensures sufficient electrical insulation. For example, the RF-capable guide wire can be one which is constructed in accordance with FIGS. 1 to 3, wherein the wire core 2 can consist in this case of a nickel titanium alloy. Alternatively, the wire core 2 can also consist of a high-strength, flexurally rigid material, such as a fiber-reinforced and in particular a carbon-fiber-reinforced plastics material. In the RF-capable guide wire, the wire core 2 can be more flexurally rigid than the multilayer shaft sheath in corresponding embodiments.

As the exemplary embodiments shown and described above make clear, the invention provides a very advantageous guide wire having a multilayer sheath of a wire core, the rigidity behavior of the guide wire being settable in a desired manner by way of said sheath. In addition, the guide wire according to the invention is highly suitable for MR applications and/or for RF applications.

1-14. (canceled)

15. A medical guide wire comprising
a wire core made of an MR-invisible material,
a sheath that surrounds the wire core at least sectionally and so as to be in touching contact therewith, and
an MR marker made of MR-visible material,
wherein the sheath has a multilayer structure which comprises at least two solid material layers and/or fiber layers which are formed by different, MR-invisible plastics materials, and
wherein the MR marker has at least one MR marker element which is integrated at least partially into the multilayer structure of the sheath or is surrounded thereby.

16. The medical guide wire as claimed in claim 15, wherein the sheath layer structure has at least two solid material layers and at least one fiber layer which is formed by a fabric material or fiber material.

17. The medical guide wire as claimed in claim 15, wherein the sheath has at least two fiber layers having at least one of different axial fiber pitches or fiber winding directions.

18. The medical guide wire as claimed in claim 15, wherein the wire core is formed as a wire strand made of a plurality of individual wires (that are connected together in a cord- or strand-forming manner.

19. The medical guide wire as claimed in claim 18, wherein the MR marker contains an MR-visible material which has been introduced into intermediate spaces between the individual wires of the wire core formed as a wire core strand.

20. The medical guide wire as claimed in claim 15, wherein the sheath surrounds the wire core at least in a shaft section adjoining a distal section, and is embodied with greater flexural rigidity than the wire core.

21. The medical guide wire as claimed in claim 15, wherein the solid material layers are manufactured from flexurally rigid plastics materials.

22. The medical guide wire as claimed in claim 21, wherein the flexurally rigid plastic materials are at least one of ABS, PEEK, PET, Ultramid, and epoxy resin materials.

23. The medical guide wire as claimed in claim 15, wherein the fiber layers are formed from at least one of glass fibers, aramid fibers, or polyester fibers.

24. The medical guide wire as claimed in claim 15, wherein the MR marker contains at least one of
an MR marker element in the region of the sheath, said MR marker element being embedded in one of the sheath layers or between two of the sheath layers or between the wire core and the adjacent sheath layer, or
an MR marker element in a distal section, free of the sheath, of the guide wire, said MR marker element being embedded in a sheath of the wire core.

25. The medical guide wire as claimed in claim 24, wherein the MR marker element contains at least one of:
MR-visible particles which are embedded in one or more of the solid material layers,
one or more MR line elements which are embedded in the sheath, as a line element that is continuous along the axial length of the sheath or as a set of one or more line elements that are axially shorter than the sheath and are arranged with or without an offset in the circumferential direction and with or without an axial offset and with identical or different lengths,
one of the fibers of the at least one fiber layer, to which end the fiber contains an MR-visible material,
one or more MR line elements which are arranged along a helical line or a line located in a longitudinal plane of the guide wire, or
one or more MR line elements which are embodied as length measuring marker elements.

26. A medical guide wire, comprising
a wire core,
a sheath that surrounds the wire core with touching contact in a shaft section,
a sleeve that surrounds the wire core with touching contact in a distal section, and
an MR-visible, wire-like or tubular auxiliary element made of a non-magnetic material, wherein the auxiliary element is arranged in a distal guide wire section and is embedded in the distal sleeve.

27. The medical guide wire as claimed in claim 26, wherein the auxiliary element comprises an auxiliary element wire which is arranged with an axially extending main component alongside the wire core or so as to surround the wire core in a helical manner.

28. The medical guide wire as claimed in claim 26, wherein the auxiliary element is doped or coated with MR marker material.

29. A medical guide wire, comprising
a wire core and
a sheath that surrounds the wire core at least sectionally and so as to be in touching contact therewith,
wherein the sheath has a multilayer, electrically insulating layer structure which has at least two solid material layers and/or fiber layers which are formed by different, electrically insulating plastics materials.
